SEQUENCE LISTING (33 sequences)

SEQ ID NO: 1 - protein, 14 aa, artificial sequence
  synthetic construct
  MOD_RES (3)..(3): cyclohexylalanine
AKXAVAAWTLKAAA

SEQ ID NO: 2 - protein, 7 aa, ARTIFICIAL SEQUENCE
  SYNTHETIC CONSTRUCT
GSGGGGS

SEQ ID NO: 3 - skipped

SEQ ID NO: 4 - protein, 16 aa, ARTIFICIAL SEQUENCE
  SYNTHETIC CONSTRUCT
  MOD_RES (1)..(1): D-alanine
  MOD_RES (3)..(3): CYCLOHEXYLALANINE
  MOD_RES (14)..(14): D-ALANINE
  MOD_RES (15)..(15): aminohexanoic acid
AKXVAAWTLKAAAAXC

SEQ ID NO: 5 - protein, 13 aa, ARTIFICIAL SEQUENCE
  SYNTHETIC CONSTRUCT
  MOD_RES (1)..(1): D-alanine
  MOD_RES (3)..(3): cyclohexylalanine
  MOD_RES (13)..(13): D-alanine
AKXVAAWTLKAAA

SEQ ID NO: 6 - protein, 13 aa, ARTIFICIAL SEQUENCE
  SYNTHETIC CONSTRUCT
  MOD_RES (1)..(1): D-alanine
  MOD_RES (13)..(13): D-alanine
AKFVAAWTLKAAA

SEQ ID NO: 7 - protein, 13 aa, ARTIFICIAL SEQUENCE
  SYNTHETIC CONSTRUCT
  MOD_RES (1)..(1): D-alanine
  MOD_RES (13)..(13): D-alanine
AKYVAAWTLKAAA

SEQ ID NO: 8 - protein, 13 aa, ARTIFICIAL SEQUENCE
  SYNTHETIC CONSTRUCT
  MOD_RES (1)..(1): D-alanine
  MOD_RES (13)..(13): D-alanine
AKFVAAYTLKAAA

SEQ ID NO: 9 - protein, 13 aa, ARTIFICIAL SEQUENCE
  SYNTHETIC CONSTRUCT
  MOD_RES (1)..(1): D-alanine
  MOD_RES (3)..(3): cyclohexylalanine
  MOD_RES (13)..(13): D-alanine
AKXVAAYTLKAAA

SEQ ID NO: 10 - protein, 13 aa, ARTIFICIAL SEQUENCE
  SYNTHETIC CONSTRUCT
  MOD_RES (1)..(1): D-alanine
  MOD_RES (13)..(13): D-alanine
AKYVAAYTLKAAA

SEQ ID NO: 11 - protein, 13 aa, ARTIFICIAL SEQUENCE
  SYNTHETIC CONSTRUCT
  MOD_RES (1)..(1): D-alanine
  MOD_RES (13)..(13): D-alanine
AKFVAAHTLKAAA

SEQ ID NO: 12 - protein, 13 aa, ARTIFICIAL SEQUENCE
  SYNTHETIC CONSTRUCT
  MOD_RES (1)..(1): D-alanine
  MOD_RES (3)..(3): cyclohexylalanine
  MOD_RES (13)..(13): D-alanine
AKXVAAHTLKAAA

SEQ ID NO: 13 - protein, 13 aa, ARTIFICIAL SEQUENCE
  SYNTHETIC CONSTRUCT
  MOD_RES (1)..(1): D-alanine
  MOD_RES (13)..(13): D-alanine
AKYVAAHTLKAAA

SEQ ID NO: 14 - protein, 13 aa, ARTIFICIAL SEQUENCE
  SYNTHETIC CONSTRUCT
  MOD_RES (1)..(1): D-alanine
  MOD_RES (13)..(13): D-alanine
AKFVAANTLKAAA

SEQ ID NO: 15 - protein, 13 aa, ARTIFICIAL SEQUENCE
  SYNTHETIC CONSTRUCT
  MOD_RES (1)..(1): D-alanine
  MOD_RES (3)..(3): cyclohexylalanine
  MOD_RES (13)..(13): D-alanine
AKXVAANTLKAAA

SEQ ID NO: 16 - protein, 13 aa, ARTIFICIAL SEQUENCE
  SYNTHETIC CONSTRUCT
  MOD_RES (1)..(1): D-alanine
  MOD_RES (13)..(13): D-alanine
AKYVAANTLKAAA

SEQ ID NO: 17 - protein, 13 aa, ARTIFICIAL SEQUENCE
  SYNTHETIC CONSTRUCT
  MOD_RES (3)..(3): cyclohexylalanine
AKXVAAWTLKAAA

SEQ ID NO: 18 - protein, 13 aa, ARTIFICIAL SEQUENCE
  SYNTHETIC CONSTRUCT
AKFVAAWTLKAAA

SEQ ID NO: 19 - protein, 13 aa, ARTIFICIAL SEQUENCE
  SYNTHETIC CONSTRUCT
AKYVAAWTLKAAA

SEQ ID NO: 20 - protein, 13 aa, ARTIFICIAL SEQUENCE
  SYNTHETIC CONSTRUCT
AKFVAAYTLKAAA

SEQ ID NO: 21 - protein, 13 aa, ARTIFICIAL SEQUENCE
  SYNTHETIC CONSTRUCT
  MOD_RES (3)..(3): cyclohexylalanine
AKXVAAYTLKAAA

SEQ ID NO: 22 - protein, 13 aa, ARTIFICIAL SEQUENCE
  SYNTHETIC CONSTRUCT
AKYVAAYTLKAAA

SEQ ID NO: 23 - protein, 13 aa, ARTIFICIAL SEQUENCE
  SYNTHETIC CONSTRUCT
AKFVAAHTLKAAA

SEQ ID NO: 24 - protein, 13 aa, ARTIFICIAL SEQUENCE
  SYNTHETIC CONSTRUCT
  MOD_RES (3)..(3): cyclohexylalanine
AKXVAAHTLKAAA

SEQ ID NO: 25 - protein, 13 aa, ARTIFICIAL SEQUENCE
  SYNTHETIC CONSTRUCT
AKYVAAHTLKAAA

SEQ ID NO: 26 - protein, 13 aa, ARTIFICIAL SEQUENCE
  SYNTHETIC CONSTRUCT
AKFVAANTLKAAA

SEQ ID NO: 27 - protein, 13 aa, ARTIFICIAL SEQUENCE
  SYNTHETIC CONSTRUCT
  MOD_RES (3)..(3): cyclohexylalanine
AKXVAANTLKAAA

SEQ ID NO: 28 - protein, 13 aa, ARTIFICIAL SEQUENCE
  SYNTHETIC CONSTRUCT
AKYVAANTLKAAA

SEQ ID NO: 29 - protein, 21 aa, ARTIFICIAL SEQUENCE
  SYNTHETIC CONSTRUCT
FNNFTVSFWLRVPKVSASHLE

SEQ ID NO: 30 - protein, 15 aa, ARTIFICIAL SEQUENCE
  SYNTHETIC CONSTRUCT
SSVFNVVNSSIGLIM

SEQ ID NO: 31 - protein, 12 aa, ARTIFICIAL SEQUENCE
  SYNTHETIC CONSTRUCT
SKMRMATPLLMQ

SEQ ID NO: 32 - protein, 15 aa, ARTIFICIAL SEQUENCE
  SYNTHETIC CONSTRUCT
QYIKANSKFIGITEL

SEQ ID NO: 33 - protein, 11 aa, PR8 virus
SFERFEIFPKE

(19) United States
(12) Patent Application Publication
(10) Pub. No.: US 2019/0184013 A1
(43) Pub. Date: Jun. 20, 2019

(54) COMPOSITIONS FOR SELECTIVE HUMORAL RESPONSES AND METHODS OF USE THEREOF

(71) Applicant: University of Miami, Miami, FL (US)

(72) Inventors: Victor L. Perez Quinones, Miami, FL (US); Pirouz Daftarian, Brisbane, CA (US)

(21) Appl. No.: 16/302,457

(22) PCT Filed: May 23, 2017
(86) PCT No.: PCT/US2017/033943

§ 371 Date: Nov. 16, 2018

Related U.S. Application Data
(60) Provisional application No. 62/340,035, filed on May 23, 2016.

(51) Int. Cl. A61K 39/395; C07K 14/47; A61P 31/00; A61P 35/00; A61P 29/00; A61P 37/00; A61K 47/50; A61K 9/51

(52) U.S. Cl. CPC A61K 39/39558; C07K 14/47; A61P 31/00; A61K 9/51; A61P 29/00; A61P 37/00; A61K 47/50; A61P 35/00

(57) ABSTRACT

Conjugates of synthetic nanocarriers, complexed with syngeneic (self) proteins adducted with haptens or other poorly immunogenic antigens (antigens of low immunogenicity), elicit selective humoral responses or antibodies against the hapten or antigen and not to self-protein. Compositions include these conjugates, which can be used as vaccines. Methods of making and using them are described herein. In a typical embodiment, a conjugate including a hapten or antigen of low immunogenicity associated with a particular disease (e.g., infection, cancer) can be used as a vaccine by eliciting antibodies that specifically neutralize the hapten or antigen. These hapten (and other poorly immunogenic antigen)-carrying nanocarriers selectively target antigen presenting cells resulting in a strong anti-hapten humoral response, and thus find use in vaccines for cancer (e.g., cancers of lung, cervix, breast, brain, liver pancreas, ovaries, skin, etc.), infectious diseases and inflammatory-mediated diseases, as well as for autoimmune disorders.

### CROSS-REFERENCE TO RELATED APPLICATION

This application claims the benefit of U.S. Provisional Application No. 62/340,035 filed May 23, 2016, the disclosure of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on May 23, 2017, is named 7230-187WO_ST25.txt and is 10 kilobytes in size.

### FIELD OF THE INVENTION

The invention relates to the fields of immunology, biochemistry and nanotechnology. More particularly, the invention relates to methods and compositions for selective elicitation of strong antibody responses against haptens and other antigens of low immunogenicity.

### BACKGROUND

A series of important antigens, known as haptens, involved in cancer, pathogens, and autoimmunity are extremely poorly immunogenic. Generation of specific antibodies against haptens has been challenging due to small molecular weight and the absence of a T helper epitope, which is required for eliciting humoral responses with high avidity. Indeed, isotype class switching and the affinity maturation of immunoglobulins is also T helper dependent. Haptens lack CD4+ T-cell epitopes, a main player for eliciting vigorous immune responses. Such epitopes stimulate specific T helper cells to provide proper cytokine milieu to support hapten-specific immune responses. CD4+ T helper epitopes, however, bind MHC class II molecules on the surface of antigen presenting cells (APCs) to initiate the cascade of mounting humoral responses. The MHC class II polymorphism is critical for epitope-based immunity and is the cause of MHC restriction. Coupling a huge carrier protein provides such T helpers for the hapten. To overcome the poor immunogenicity of haptens, non-self protein carriers are used in traditional hapten-carrier conjugates mainly because they provide the T helper epitopes that haptens lack. Such carriers have serious limitations and are known to lack in producing highly specified anti-hapten antibodies (Renjifo X et al., Journal of Immunology 1998, 161(2): 702-6; Herzenberg L A et al., Nature 1980, 285(5767): 664-7; Jegerlehner A et al., Vaccine 2010, 28(33): 5503-12).

### SUMMARY

Described herein are conjugates of synthetic nanocarriers, complexed with syngeneic (self) proteins adducted with haptens or other poorly immunogenic antigens (antigens of low immunogenicity), for eliciting (producing) selective humoral responses or antibodies against the hapten or antigen and not to self-protein. Compositions including these conjugates, which can be used as vaccines, and methods of making and using them, are described herein. In a typical embodiment, a conjugate including a hapten or antigen of low immunogenicity associated with a particular disease (e.g., infection, cancer) can be used as a vaccine by eliciting antibodies that specifically neutralize the hapten or antigen. The data described herein shows that a novel PADRE-Derived-Dendrimer system (PDD) delivers haptens (poor antigens) selectively to APCs eliciting strong humoral immunity. A hapten notorious for poor immunogenicity, 2-(ω-carboxyethyl)pyrrole (CEP), was coupled to mouse serum albumin (MSA) and was complexed with PDD Immunization of C57BL/6 mice with the PDD/CEP-MSA complex elicited high titers of anti-CEP with no additional adjuvant. Antibody levels as measured by ODs were significantly higher than those elicited by conventional CEP complexes with non-self-protein carrier keyhole limpet hemocyanin (CEP-KLH) and adjuvant (Titermax) immunizations. Labeled PDD/CEP-MSA was shown to target both murine and human APCs in vitro as well as murine APCs in vivo. Furthermore, the anti-CEP elicited by PDD/CEP has significantly higher specificity with no activity against the self-carrier proteins, like albumin. From mice immunized with PDD/CEP-MSA, two highly specific monoclonal anti-CEP clones were generated. Characterization of the selected clones revealed that they were reactive to human-serum-albumin-CEP (HSA-CEP) and CEP-KLH, but not the protein carriers, albumin or KLH. PDD/CEP-MSA immunized sera did not show reactivity to any structures similar to CEP or 2-(ω-carboxypropyl)pyrrole (CPP) coupled to MSA (CPP-MSA). The data revealed that the PDD/haptenated-self-protein platform was able to elicit a strong anti-hapten humoral response and serve as a tool to make monoclonal antibodies against poorly immunogenic antigens and haptens. These hapten (and other poorly immunogenic antigen)-carrying nanocarriers selectively target APCs resulting in a strong anti-hapten humoral response, and thus find use in vaccines for cancer (e.g., cancers of lung, cervix, breast, brain, liver pancreas, ovaries, skin, etc.), infectious diseases and inflammatory-mediated diseases, as well as for autoimmune disorders.

Accordingly, described herein is a conjugate including at least one charged dendrimer having conjugated thereto: a) at least one T helper peptide that specifically binds to a professional APC, b) at least one hapten or antigen of low immunogenicity, and c) at least one syngeneic peptide or protein. The subject can be, for example, a mammal. The at least one T helper peptide can be a Pan-DR epitope (PADRE). The at least one T helper peptide can include the amino acid sequence of any of SEQ ID NOs: 1-33 or a derivative thereof. The at least one charged dendrimer can be a PAMAM dendrimer. The syngeneic peptide or protein can be, for example, serum albumin.

Also described herein is a method of producing antibodies against a hapten or antigen of low immunogenicity in a subject. The method includes the steps of: immunizing the subject with a conjugate as described herein resulting in antibodies specific for the at least one hapten or antigen of low immunogenicity; and isolating the antibodies (e.g., polyclonal antibodies).

Further described herein is a method of producing monoclonal antibodies against a hapten or antigen of low immunogenicity in a subject. The method includes immunizing the subject with a conjugate as described herein resulting in reactive B cells for making monoclonal antibodies via fusions and generation of hybridomas, via phage display technology, or via any manipulation of B cell nucleic acids.

Yet further described herein is a method of increasing immunogenicity of a hapten or antigen of low immunogenicity in a subject. The method includes conjugating the hapten or antigen of low immunogenicity to a charged dendrimer having conjugated thereto: a) at least one T helper peptide that specifically binds to a professional APC, and b) at least one syngeneic peptide or protein.

Still further described herein is a vaccine for eliciting a humoral response against a hapten or antigen of low immunogenicity in a subject. The vaccine includes a conjugate as described herein and a pharmaceutically acceptable carrier.

Also described herein is a kit for generating antibodies against a hapten or antigen of low immunogenicity. The kit includes a plurality of conjugates, each conjugate including at least one charged dendrimer having conjugated thereto: a) at least one T helper peptide that specifically binds to a professional APC, b) at least one hapten or antigen of low immunogenicity, and c) at least one syngeneic peptide or protein; instructions for use; and packaging.

Unless otherwise defined, all technical terms used herein have the same meaning as commonly understood by one of ordinary skill in the art to which this invention belongs.

As used herein, a “nucleic acid” or a “nucleic acid molecule” means a chain of two or more nucleotides such as RNA (ribonucleic acid) and DNA (deoxyribonucleic acid), and chemically-modified nucleotides. A “purified” nucleic acid molecule is one that is substantially separated from other nucleic acid sequences in a cell or organism in which the nucleic acid naturally occurs (e.g., 30, 40, 50, 60, 70, 80, 90, 95, 96, 97, 98, 99, 100% free of contaminants). The terms include, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced by polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

When referring to an amino acid residue in a peptide, oligopeptide or protein, the terms “amino acid residue”, “amino acid” and “residue” are used interchangeably and, as used herein, mean an amino acid or amino acid mimetic joined covalently to at least one other amino acid or amino acid mimetic through an amide bond or amide bond mimetic.

As used herein, “protein” and “polypeptide” are used synonymously to mean any peptide-linked chain of amino acids, regardless of length or post-translational modification, e.g., glycosylation or phosphorylation.

When referring to a nucleic acid molecule, polypeptide, or infectious pathogen, the term “native” refers to a naturally-occurring (e.g., a wild-type (WT)) nucleic acid, polypeptide, or infectious pathogen.

As used herein, the terms “antigen” and “immunogen” mean a molecule that is specifically recognized and bound by an antibody. The terms “antigen of low immunogenicity” and “poorly immunogenic antigen” are used interchangeably herein and mean an antigen that when injected into a host, has a low ability or no ability to elicit immune responses (e.g. antibody responses) against itself. Poor or low immunogenicity may be a result of the size of the antigen being less than 1000 Dalton, having a simple structure, being conserved in many species, or the absence of immunological epitopes (which are needed for the immune system to sense them and respond to them). Among examples of poor antigens are glycolipids. Just to name one as an example, GD2 is a poor antigen and is a disialoganglioside involved in cell growth and differentiation, which is highly expressed on neuroblastoma, melanoma, glioma, and small-cell lung cancer. Another example is CEP.

When referring to an epitope (e.g., T helper epitope, T helper peptide), by biological activity is meant the ability to bind an appropriate MHC molecule and, in the case of peptides useful for stimulating CTL responses, induce a T helper response and a CTL response against a target antigen or antigen mimetic.

A “T helper peptide” as used herein refers to a peptide recognized by the T cell receptor of T helper cells. For example, the PADRE peptides described herein are T helper peptides. A T helper peptide is an example of an epitope, e.g., a t helper epitope.

When referring to PDD, other conjugates and dendrimers, by the term “cargo” is meant any entity that is carried by a PDD, other conjugate or dendrimer. The term can include a hapten alone or a hapten(s) combined with a carrier such as (bovine serum) albumin, Keyhole limpet hemocyanin (KLH), cryoglobulin, polyethylene glycol (PEG) polymer, etc.

The terms “specific binding” and “specifically binds” refer to that binding which occurs between such paired species as enzyme/substrate, receptor/agonist, antibody/antigen, etc., and which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs.

As used herein, the terms “Pan-DR epitope,” “Pan DR T helper epitope,” “Pan-HLA-DR-binding epitope,” “PADRE” and “PADRE peptides” mean a peptide of between about 4 and about 20 residues that is capable of binding at least about 7 of the 12 most common DR alleles (DR1, 2w2b, 2w2a, 3, 4w4, 4w14, 5, 7, 52a, 52b, 52c, and 53) with high affinity. “High affinity” is defined herein as binding with an IC₅₀% of less than 200 nm. For example, high affinity binding includes binding with an IC₅₀% of less than 3100 nM. For binding to Class II MHC, a binding affinity threshold of 1,000 nm is typical, and a binding affinity of less than 100 nm is generally considered high affinity binding. Construction and use of PADRE peptides is described in detail in U.S. Pat. No. 5,736,142 which is incorporated herein by reference. A list of several examples of PADRE sequences is included below.

As used herein, the terms “MHC class II” and “MHC II” mean major histocompatibility complex class II. In humans, MHC class II are also called “HLA-DR.”

By the terms “MHC II targeting peptide” and “MHC class II targeting peptide” is meant any peptide that binds to an MHC class II molecule or domain thereof.

As used herein, the term “dendrimer” means a charged (e.g., positively-charged, negatively-charged) substantially spherical or substantially linear polymer or macromolecule ranging from approximately 5 nm to approximately 50 nm. An example of a dendrimer is a charged, highly branched polymeric macromolecule with roughly spherical shape. Such a dendrimer can be, for example, a positively-charged, highly branched polymeric PAMAM dendrimer. In a specific embodiment, a dendrimer is a highly branched macromolecule spanning from a central core and containing a series of layers, structurally and synthetically distinct, which are usually referred to as ‘generations’.

When referring to a dendrimer, by the phrase “highly branched” is meant a polymer with branched architecture with a high number of functional groups.

By the terms “PAMAM dendrimer” and “poly-amidoamine dendrimer” is meant a type of dendrimer in which tertiary amines are located at branching points and connections between structural layers are made by amide functional groups. PAMAM dendrimers exhibit many positive charges on their surfaces. PAMAM with many different surface groups, e.g., amidoethanol, midoethylethanolamine, amino, succinamic acid, hexlamide, etc., are commercially available.

By the term “derivatized dendrimer” is meant a dendrimer having one or more functional groups conjugated to its surface.

A “PADRE-derivatized dendrimer,” “PDD” or “PADRE-dendrimer” is a dendrimer with one or more PADRE peptides covalently attached thereto (e.g., to the functional groups on the surface of a dendrimer).

As used herein, the terms “professional antigen presenting cell” and “PAPC” mean cells that displays foreign antigens in the context of self MHC on their surfaces and includes dendritic cells, macrophages, monocytes, and B cells.

By the term “conjugated” is meant when one molecule or agent is physically or chemically coupled or adhered to another molecule or agent. Examples of conjugation include covalent linkage (e.g., covalently bound drug or other small molecule) and electrostatic complexation. The terms “complexed,” “complexed with,” and “conjugated” are used interchangeably herein.

As used herein, the phrase “sequence identity” means the percentage of identical subunits at corresponding positions in two sequences (e.g., nucleic acid sequences, amino acid sequences) when the two sequences are aligned to maximize subunit matching, i.e., taking into account gaps and insertions. Sequence identity can be measured using sequence analysis software (e.g., Sequence Analysis Software Package from Accelrys CGC, San Diego, Calif.).

The phrases “isolated” or “biologically pure” refer to material which is substantially or essentially free from components which normally accompany it as found in its native state.

As used herein, the terms “nanoparticle,” “nanovehicle” and “nanocarrier” mean a microscopic particle whose size is measured in nanometers. In one example, a nanoparticle, nanovehicle or nanocarrier is a PDD or a particle combining several PADRE-dendrimer conjugates with a total diameter in the range of approximately 2-500 nm.

As used herein, the term “net-charge” means the sum of the electric charges of the particles or compounds in a physiological pH.

As used herein, the term “therapeutic agent” is meant to encompass any molecule, chemical entity, composition, drug, or biological agent capable of curing, healing, alleviating, relieving, altering, remedying, ameliorating, improving or affecting a disease, the symptoms of disease, or the predisposition toward disease. The term “therapeutic agent” includes small molecules, antisense reagents, nucleic acids, siRNA reagents, antibodies, enzymes, polypeptides, peptides, organic or inorganic molecules, natural or synthetic compounds and the like.

The term “antibody” is meant to include polyclonal antibodies, monoclonal antibodies (mAbs), chimeric antibodies, humanized antibodies, anti-idiotypic (anti-Id) antibodies to antibodies that can be labeled in soluble or bound form, as well as fragments, regions or derivatives thereof, provided by any known technique, such as, but not limited to, enzymatic cleavage, peptide synthesis or recombinant techniques.

As used herein the term “adjuvant” means any material or substance which enhances the humoral and/or cellular immune response.

As used herein, the terms “displayed” or “surface exposed” are considered to be synonyms, and refer to antigens or other molecules that are present (e.g., accessible to immune site recognition) at the external surface of a structure such as a nanoparticle or nanocarrier (e.g., PADRE-dendrimer, HA-dendrimer, etc.).

As used herein, “vaccine” includes all prophylactic and therapeutic vaccines. The vaccine compositions described herein are suitable for administration to subjects in a biologically compatible form in vivo. The expression “biologically compatible form suitable for administration in vivo” as used herein means a form of the substance to be administered in which any toxic effects are outweighed by the therapeutic effects. The substances may be administered to any animal, e.g., humans. In some embodiments, a vaccine as described herein is administered to a mammal, e.g., a rodent or rabbit, for producing monoclonal antibodies against a particular antigen.

By the phrase “immune response” is meant induction of antibody and/or immune cell-mediated responses specific against an antigen, antigens, pathogen, pathogenic agent, etc. An immune response has many facets, some of which are exhibited by the cells of the immune system (e.g., B-lymphocytes, T-lymphocytes, macrophages, and plasma cells). Immune system cells may participate in the immune response through interaction with an antigen or pathogen or other cells of the immune system, the release of cytokines and reactivity to those cytokines. Immune responses are generally divided into two main categories—humoral and cell-mediated. The humoral component of the immune response includes production of antibodies specific for an antigen or pathogen. The cell-mediated component includes the generation of delayed-type hypersensitivity and cytotoxic effector cells against the antigen or pathogen. An immune response can include, for example, activation of a CD4 T helper response.

By the phrases “therapeutically effective amount” and “effective dosage” is meant an amount sufficient to produce a therapeutically (e.g., clinically) desirable result; the exact nature of the result will vary depending on the nature of the disorder being treated. For example, where the disorder to be treated is cancer, the result can be elimination of cancer cells, a reduction in growth of cancer cells, a reduction in size or elimination of a tumor associated with the cancer, etc. As another example, where the disorder to be treated is a pathogenic infection, the result can be elimination of the pathogen, a reduction in growth of the pathogen, a reduction in size or elimination of a lesion associated with the pathogen, etc. The compositions, conjugates, vaccines and nanocarriers described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions, conjugates, vaccines and nanocarriers described herein can include a single treatment or a series of treatments.

As used herein, the term “treatment” is defined as the application or administration of a therapeutic agent described herein, or identified by a method described herein, to a patient or subject or individual, or application or administration of the therapeutic agent to an isolated tissue or cell line from a patient, subject or individual who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean an animal to be treated, including vertebrates and invertebrates. Typically, a subject is a human. In some cases, the methods of the invention find use in experimental animals, in veterinary applications (e.g., equine, bovine, ovine, canine, feline, avian, etc.), and in the development of animal models for disease, including, but not limited to, rodents including mice, rats, and hamsters, as well as non-human primates.

Although compositions, conjugates, vaccines, kits, and methods similar or equivalent to those described herein can be used in the practice or testing of the present invention, suitable compositions, conjugates, vaccines, kits, and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a graph and a chart showing results from Dynamic Light Scattering Characterization of PDD/CEP-MSA complex. The average diameter, polydispersity, and zeta potential of the PDD//CEP-MSA complex were determined by means of dynamic light scattering. The data are the average+/−standard deviation of separate experiments.

FIG. 2 is a pair of plots showing the flow cytometry data of the complex of PDD/CEP-MSA-FITC made of a 7/1 ratio.

FIGS. 3A, 3B, 3C and 3D are a series of graphs showing a comparison of efficacy of MSA versus Titermax/CEP-vaccines for eliciting anti-CEP antibodies in mice. Analysis of anti-CEP detection in the sera of mice vaccinated with PDD/CEP-MSA complex or Titermax/KLH-CEP were performed. Groups of mice received two vaccinations with either 20 ug of CEP-MSA formulated in PDD or 50 ug of KLH-CEP emulsified in Titermax, via s.c. injections. Mice were bled 10 days post last immunizations and anti-CEP titers were determined by ELISA.

FIGS. 4A and 4B are images showing in vitro and in vivo delivery of MSA-CEP-FITC without or with PDD. FIG. 4A. Murine macrophages were co-cultured with MSA-CEP-FITC or PDD/MSA-CEP-FITC and cells were washed and imaged by fluorescent microscopy in 2 hours. FIG. 4B. In vivo delivery of PDD/MSA-CEP-FITC complex to spleens of mice. Splenocytes are imaged by fluorescent microscopy 12 hours post-iv injection of MDA-CEP-FITC (Left Panel) or PDD/MDA-CEP-FITC (Right Panel). Mice in groups of three received 20 ug of formulations of MDA-CEP-FITC alone or complexed with PDD in saline. Representative images are shown.

### DETAILED DESCRIPTION

Described herein is a derivatized dendrimer vaccine platform that can be used to elicit highly specific anti-hapten (anti-antigen) antibody responses. This platform negates the use of non-self immunogenic carriers, avoiding unwanted adverse reactions, and has an APC-targeting ability that generates higher value hapten-specific antibodies with higher specificity while lowering the dose and the frequency of immunizations. The increased immunogenicity achieved by preferential targeting of APCs and strong adjuvant activity of universal peptide binding MHC II is implemented to elicit antibody responses against antigens with low immunogenicity including haptens. In order to develop monoclonal antibodies with high specificity against a hapten with poor immunogenicity, a challenging antigen with high clinical importance was selected and tested. Protein adducts of 2-ω-carboxyethylpyrrole (CEP) have gained much attention recently since they have been linked to a variety of pathologic processes including age related macular degeneration (AMD), cancer, Autism, and wound healing. Oxidation of docosahexaenoyl phospholipids after binding with proteins and oxidative lipolysis can generate CEP-modified protein. CEP-modified protein generated due to oxidation in outer segments of photoreceptors was found to be elevated significantly in the retina and blood of AMD patients. Also, autoantibodies against these CEP-modified proteins were found to be increased in AMD patients' plasma. CEP-modified protein was also seen in neurofilaments of brains in autistic cases, appearing to be a hallmark of autistic brain and it is confirming evidence for the role of oxidative stress as one of the potential causes of autism. Better understanding of the CEP role in these pathologic conditions is pivotal for discovery of disease biomarkers and drug and development. Therefore generation of selective monoclonal antibodies against CEP is essential for conducting such studies. Also, generation of specific antibodies against CEP has been challenging due to its small molecular weight of approximately 270 Daltons and the absence of a T helper epitope, which is required for eliciting humoral responses with high avidity and affinity. Likewise, isotype class switching and the affinity maturation of immunoglubulins is also T helper dependent. Production of a specific antibody against CEP is challenging since, in theory, it should shape epitopes with random neighboring amino acids on carrier proteins. Also the specific anti-CEP antibody should be able to discriminate CPP despite their close chemical similarity. Since PDD contains a promiscuous T helper epitope, it was postulated that it should provide sufficient help negating a need for a non-self carrier protein. Furthermore, since PDD has tropism for APC tropic, it reduces the off targeting vaccine delivery. In the experiments described herein, complexes of PDD with a syngeneic (self) protein loaded with a hapten served as a simple template to make anti-hapten immune responses. The use of an adjuvanated/APC targeting nanocarrier that hosts a self-albumin CEP adduct to mount antibody responses only against the hapten moiety was demonstrated.

The below described preferred embodiments illustrate adaptations of these compositions, conjugates, vaccines, kits, platforms and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates) Immunology techniques are generally known in the art and are described in detail in methodology treatises such as Advances in Immunology, volume 93, ed. Frederick W. Alt, Academic Press, Burlington, Mass., 2007; Making and Using Antibodies: A Practical Handbook, eds. Gary C. Howard and Matthew R. Kaser, CRC Press, Boca Raton, Fla., 2006; Medical Immunology, 6^{th} ed., edited by Gabriel Virella, Informa Healthcare Press, London, England, 2007; and Harlow and Lane ANTIBODIES: A Laboratory Manual, Cold Spring Harbor Laboratory Press, Cold Spring Harbor, N.Y., 1988. Construction and use of PAMAM dendrimers is also described, for example, in U.S. patent application Ser. Nos. 13/262,285 and 13/321,521; Arashkia et al., Virus Genes 40 (1): 44-52, 2010; Velders et al., J Immunol. 166:5366-5373, 2001; and S. Chauhan, N. K. Jain, P. V. Diwan. (2009) Pre-clinical and behavioural toxicity profile of PAMAM dendrimers in mice. Proceedings of the Royal Society A: Mathematical, Physical and Engineering Sciences (Online publication date: Dec. 3, 2009).

### Conjugates and Vaccines for Eliciting a Humoral Response Against a Hapten or Antigen of Low Immunogenicity

A conjugate for eliciting a humoral response against a hapten or antigen of low immunogenicity in a subject (e.g., a mammal) includes at least one charged dendrimer having conjugated thereto: a) at least one T helper peptide that specifically binds to a PAPC, b) at least one hapten or antigen of low immunogenicity, and c) at least one syngeneic peptide or protein. In some cases, the hapten is not a peptide. The at least one charged dendrimer can be any suitable charged dendrimer, such as, for example, a PAMAM dendrimer. Additional types of dendrimers are discussed below. The syngeneic peptide or protein can be any suitable syngeneic peptide or protein, such as, for example, serum albumin. The choice of syngeneic peptide or protein depends upon the size ideally bigger than 20,000 Dalton, cost and availability of pure material.

The at least one T helper peptide can be any suitable T helper peptide. Several examples of T helper peptides are set forth in SEQ ID Nos: 1-33. In one embodiment, the at least one T helper peptide that specifically binds to a PAPC is a Pan-DR epitope, e.g., PADRE. PADRE is an artificially designed peptide that binds to the majority of murine and human MHC Class II molecules, and conjugating PADRE peptides to dendrimers (e.g., a PDD) makes the resultant complex or conjugate a ligand for PAPCs that express high levels of MHC class II. PADRE is a synthetic, non-natural T helper epitope [AKchxAVAAWTLKAAA (chxA=cyclohexylalanine) (SEQ ID NO: 1)]. When fused to the surface of the dendrimer, PADRE will bind and activate primarily cells that have MHC class II including all PAPCs. Several PADRE peptides (e.g., 2, 3, 4, 5, etc.) can be attached to each dendrimer. The attachment may be done without or with suitable spacers to preserve the binding properties of the peptide that give rise to its immunogenic properties. Spacers may be any combination of amino acids including AAA, KK, GS, GSGGGGS (SEQ ID NO: 2), RS, or AAY. As used herein, the terms “linker” or “spacer” mean the chemical groups that are interposed between the dendrimer and the surface exposed molecule(s) such as the AAA that is conjugated or bound to the dendrimer (e.g., PADRE-dendrimer) and the surface exposed molecule(s). Preferably, linkers are conjugated to the surface molecule at one end and at their other end to the nanoparticle (e.g., PADRE-dendrimer). Linking may be performed with either homo- or heterobifunctional agents, i.e., SPDP, DSS, SIAB. Methods for linking are disclosed in PCT/DK00/00531 (WO 01/22995) to deJongh, et al., which is hereby incorporated by reference in its entirety. In another embodiment, the at least one T helper peptide that specifically binds to PAPCs is influenza HA. Typically, the at least one T helper peptide is a T helper epitope or any other epitope that activates or contributes to activation of CD4+T helper cells. T helper epitope activation of CD4+T helper cells is required for the expansion and stimulation of CD8 T cells as well as for antibody production by B cells, both of which are essential for induction of protective immune responses against infectious agents, cancer, inflammatory-mediated diseases, auto-immune disorders, etc.

Compositions including a conjugate are described herein, and can include a plurality of conjugates and a pharmaceutically acceptable carrier. The compositions and conjugates described herein can be used as vaccines for eliciting a humoral response against a hapten or other antigen of low immunogenicity in a subject. Such vaccines are useful for cancer, infectious diseases and inflammatory-mediated diseases, as well as for autoimmune disorders. With regard to cancer, the vaccines can be used to treat or prevent any type of cancer, including, as examples, cancers of the lung, cervix, breast, brain, liver pancreas, ovaries, and skin. With regard to infectious diseases, examples of pathogens include but are not limited to, pathogenic parasitic, bacterial, fungal, and viral organisms.

The conjugates, vaccines and compositions described herein have both prophylactic and treatment applications, i.e., can be used as a prophylactic to prevent onset of a disease or condition in a subject, as well as to treat a subject having a disease or condition. For example, a composition (e.g., vaccine) as described herein can be used to reduce the growth of or eliminate cancer cells. As another example, a composition as described herein can be used to reduce the growth of or eliminate any infectious pathogen, as well as mount an immune response against any infectious pathogen preventing an infection.

### Synthesis of Conjugates

Described herein are dendrimers having conjugated thereto at least one T helper peptide, at least one hapten or antigen of low immunogenicity, and at least one syngeneic peptide or protein (conjugates). Dendrimers can be prepared and conjugated to a T helper peptide (e.g., an epitope such as the PADRE peptide or Influenza HA) and bound to or complexed with a hapten (or other poorly immunogenic antigen) and at least one syngeneic peptide or protein using any suitable method. Methods of producing and using dendrimers (e.g., PAMAM dendrimers) are well known in the art and are described, for example, in U.S. patent application Ser. Nos. 13/262,285 and 13/321,521, Zhang J-T et. al. Macromol. Biosci. 2004, 4, 575-578, and U.S. Pat. Nos. 4,216,171 and 5,795,582. See also: D. A. Tomalia, A. M. Naylor, and W. A. Goddard III, “Starburst Dendrimers: Molecular-Level Control of Size, Shape, Surface Chemistry, Topology, and Flexibility from Atoms to Macroscopic Matter”, Angew. Chem. Int. Ed. Engl. 29 (1990), 138-175. In the experiments described herein, PAMAM dendrimers were used. However, any suitable positively charged, highly branched polymeric dendrimer can be used. Examples of additional positively charged, highly branched polymeric dendrimers include poly(propylene imine) (PPI) dendrimers or, more generally, any other dendrimers with primary amine groups on their surfaces.

In one embodiment, dendrimers are conjugated to at least one PADRE peptide (e.g., 2, 3, 4, 5, etc.), at least one hapten or antigen of low immunogenicity, and at least one syngeneic peptide or protein. The hapten may be directly conjugated to the PDD or alternatively, be covalently coupled to a carrier. In the latter case, the carrier-hapten may be noncovalently complex with PDD, e.g. based on the opposite charges. The PDD described herein can be prepared by any suitable method. Methods of making and using PADRE are known in the art. See, for example, U.S. Pat. No. 5,736,142 and U.S. patent application Ser. Nos. 13/262,285 and 13/321,521, and can be prepared according to the methods described therein, for example, or they can be purchased (e.g., from Anaspec, Inc., Fremont, Calif.). Because of their relatively short size, the PADRE peptides can be synthesized in solution or on a solid support in accordance with conventional techniques. Various automatic synthesizers are commercially available and can be used in accordance with known protocols. Alternatively, recombinant DNA technology may be employed wherein a nucleotide sequence which encodes a T helper peptide is inserted into an expression vector, transformed or transfected into an appropriate host cell and cultivated under conditions suitable for expression. These procedures are generally known in the art, as described generally in Sambrook et al., (supra), which is incorporated herein by reference. PADRE peptides as described herein may include modifications to the N- and C-terminal residues. As will be well understood by the artisan, the N- and C-termini may be modified to alter physical or chemical properties of the peptide, such as, for example, to affect binding, stability, bioavailability, ease of linking, and the like. The PADRE peptides described herein may be modified in any number of ways to provide desired attributes, e.g., improved pharmacological characteristics, while retaining substantially all of the biological activity of the unmodified peptide.

In the experiments described herein, the PADRE-dendrimer conjugate was made by simple amide coupling between the —COOH terminus of the PADRE peptide and one of the dendrimer amine groups. The PADRE peptide (Ac-D-Ala-Lys-Cha-Val-Ala-Ala-Trp-Thr-Leu-Lys-Ala-Ala-Ala-D-Ala-Ahx-Cys-OH (SEQ ID NO: 4)) (Ac=acetylated; D-Ala=D-alanine; Cha=cyclohexylalanine; Ahx=aminohexanoic acid) was purchased from Twentyfirst Century Biochemicals, Inc. (Marlboro, Mass.) in its acetylated form in order to protect the amine terminus and prevent its reaction. The purchased peptide had a minimum purity of 95%. The amide coupling reaction was carried out under standard conditions in DMF solution or in MBS. There are variants of PADRE, and all such variants are encompassed by the compositions, conjugates, vaccines, and methods described herein. For example, the PADRE peptide variants including aKXVAAWTLKAAa (SEQ ID NO: 5) bind with high or intermediate affinity (IC₅₀<1,000 nM) to 15 out of 16 of the most prevalent HLA-DR molecules ((Kawashima et al., Human Immunology 59:1-14 (1998); Alexander et al., Immunity 1:751-761 (1994)). However, other peptides which also can bind MHC class II and activate CD4 T helper cells in most humans may also be used to tag the dendrimer.

Examples of T helper peptides (e.g., APC targeting peptides) include but are not limited to: tetanus toxoid (TT) peptide 830-843; the “universal” epitope described in Panina-Bordignon et al., (Eur. J. Immunology 19:2237-2242 (1989)); and the following peptides that react with MHC class II of most human HLA, and many of mice: aKFVAAWTLKAAa (SEQ ID NO: 6), aKYVAAWTLKAAa (SEQ ID NO: 7), aKFVAAYTLKAAa (SEQ ID NO: 8), aKXVAAYTLKAAa (SEQ ID NO: 9), aKYVAAYTLKAAa (SEQ ID NO: 10), aKFVAAHTLKAAa (SEQ ID NO: 11), aKXVAAHTLKAAa (SEQ ID NO: 12), aKYVAAHTLKAAa (SEQ ID NO: 13), aKFVAANTLKAAa (SEQ ID NO: 14), aKXVAANTLKAAa (SEQ ID NO: 15), aKYVAANTLKAAa (SEQ ID NO: 16), AKXVAAWTLKAAA (SEQ ID NO: 17), AKFVAAWTLKAAA (SEQ ID NO: 18), AKYVAAWTLKAAA (SEQ ID NO: 19), AKFVAAYTLKAAA (SEQ ID NO: 20), AKXVAAYTLKAAA (SEQ ID NO: 21), AKYVAAYTLKAAA (SEQ ID NO: 22), AKFVAAHTLKAAA (SEQ ID NO: 23), AKXVAAHTLKAAA (SEQ ID NO: 24), AKYVAAHTLKAAA (SEQ ID NO: 25), AKFVAANTLKAAA (SEQ ID NO: 26), AKXVAANTLKAAA (SEQ ID NO: 27), AKYVAANTLKAAA (SEQ ID NO: 28), FNNFTVSFWLRVPKVSASHLE (SEQ ID NO: 29), SSVFNVVNSSIGLIM (SEQ ID NO: 30), SKMRMATPLLMQ (SEQ ID NO: 31), and QYIKANSKFIGITEL (SEQ ID NO: 32), (a=D-alanine, X=cyclohexylalanine). Such peptides bind to MHC class II molecules present on T cells of more than 95% of all humans. Another example of an epitope that may be used is the HA peptide sequence SFERFEIFPKE (SEQ ID NO:33) (from the provirus PR8 virus HA) that binds to mouse Balb/c MHC classII IaD.

Generally, generation-5 (G5) dendrimers are used in the compositions, conjugates, vaccines, kits, platforms and methods described herein. However, other generation dendrimers (see Table 1) can be used.

TABLE 1
PAMAM Dendrimers

| Generation | Molecular Weight | Diameter (nm) | Surface Groups |
|---|---|---|---|
| 0 | 517 | 1.5 | 4 |
| 1 | 1,430 | 2.2 | 8 |
| 2 | 3,256 | 2.9 | 16 |
| 3 | 6,909 | 3.6 | 32 |
| 4 | 14,215 | 4.5 | 64 |
| 5 | 28,826 | 5.4 | 128 |
| 6 | 58,0548 | 6.7 | 256 |

### Methods of Producing Antibodies Against a Hapten or Poorly Immunogenic Antigen

One embodiment of a method of producing antibodies against a hapten or antigen of low immunogenicity in a subject includes the steps of: immunizing the subject with a conjugate as described herein resulting in antibodies specific for the at least one hapten or antigen of low immunogenicity; and isolating the antibodies. In a typical embodiment, the antibodies are polyclonal antibodies. In the method, any suitable known techniques and protocols for isolating the antibodies can be used.

In another embodiment, a method of producing monoclonal antibodies against a hapten or antigen of low immunogenicity in a subject includes immunizing the subject with a conjugate as described herein resulting in reactive B cells for making monoclonal antibodies via any suitable methods. Suitable techniques and protocols for producing monoclonal antibodies are known, and include fusions and generation of hybridomas, phage display technology, and manipulation of B cell nucleic acids.

### Kits for Producing Antibodies Against a Hapten or Poorly Immunogenic Antigen

A kit for generating antibodies against a hapten or antigen of low immunogenicity includes a plurality of conjugates as described herein, instructions for use, and packaging. A typical kit includes a container that includes a plurality of conjugates as described herein (e.g., PDD, dendrimers conjugated to influenza HA, etc.), and a physiological buffer. Instructional materials for preparation and use of the conjugates described herein are generally included. While the instructional materials typically include written or printed materials, they are not limited to such. Any medium capable of storing such instructions and communicating them to an end user is encompassed by the kits herein. Such media include, but are not limited to electronic storage media (e.g., magnetic discs, tapes, cartridges, chips), optical media (e.g., CD ROM), and the like. Such media may include addresses to internet sites that provide such instructional materials.

### Administration of Compositions

The vaccines, conjugates and compositions described herein may be administered to animals, including vertebrates, invertebrates, and mammals (e.g., dog, cat, pig, horse, rodent, non-human primate, human), in any suitable formulation. For example, a conjugate as described herein may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions, conjugates and vaccines described herein may be administered to mammals by any conventional technique. Typically, such administration will be parenteral (e.g., intravenous, subcutaneous, intratumoral, intramuscular, intraperitoneal, or intrathecal introduction). The compositions may also be administered directly to a target site. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form. In therapeutic applications, the compositions and vaccines described herein are administered to an individual already suffering from cancer, autoimmune disease, inflammatory disease, or infected with the pathogen (e.g., virus) of interest. In prophylactic applications, the compositions and vaccines described herein are administered to an individual at risk of developing (e.g., genetically predisposed to, or environmentally exposed to) a disease or disorder, e.g., cancer, an infectious disease (i.e., infected with a pathogen (e.g., virus) of interest), an autoimmune disorder, inflammatory disease, etc.

### Effective Doses

The vaccines, conjugates and compositions described herein are preferably administered to an animal (e.g., a mammal such as a dog, cat, pig, horse, rodent, non-human primate, human) in an effective amount, that is, an amount capable of producing a desirable result in a treated animal (e.g., prevention or elimination of cancer in a mammal, protection against infectious disease(s), inflammatory disease, autoimmune disease, etc.). Such a therapeutically effective amount can be determined as described below.

Toxicity and therapeutic efficacy of the vaccines, conjugates and compositions described herein can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

Therapeutically effective amounts of the compositions, conjugates and vaccines described herein generally range for the initial immunization (that is for therapeutic or prophylactic administration) from about 1 μg to about 25,000 μg (e.g., 1, 100, 500, 2000, 2500, 10,000, 15,000, 25,000 μg) of a complex of T helper peptide/dendrimer conjugated to a hapten/poorly immunogenic antigen and a syngeneic peptide for a 70 kg patient, followed by boosting dosages of from about 1 μg to about 2500 μg of the complex (vaccine) pursuant to a boosting regimen over weeks to months depending upon the patient's response and condition by measuring specific CTL activity and/or antibody responses in the patient's blood. In one embodiment, 15 daily administrations of dendrimer in doses >133-fold greater then the above doses may be administered to a mammal with no toxicity (see Abhay Singh Chauhan et. al. 2009 Proc. R. Soc. A, 466, pp 1535-1550.2009).

For treating a subject currently suffering from cancer, inflammatory disease, an autoimmune disorder or an infectious disease and/or who has just been diagnosed with such a disease, administration preferably begins at the first sign of disease or the detection or surgical removal of tumors or shortly after diagnosis in the case of acute infection. This is followed by boosting doses until at least symptoms are substantially abated and for a period thereafter. In chronic infection, loading doses followed by boosting doses may be required.

For prophylactic use, administration may begin as soon as an individual becomes aware of a predisposition to a disease (e.g., cancer), or prior to an expected exposure to an infectious disease or pathogenic agent.

As is well known in the medical and veterinary arts, dosage for any one subject depends on many factors, including the subject's size, body surface area, age, the particular composition to be administered, time and route of administration, general health, and other drugs being administered concurrently. Several recent clinical trials testing dendrimers have examined different doses and routes of administration for safety and enhanced immunogenicity; general safety and enhanced immunogenicity have been repeatedly reported and established.

### EXAMPLES

The present invention is further illustrated by the following specific examples. The examples are provided for illustration only and should not be construed as limiting the scope of the invention in any way.

### Example 1—Characterization of PDD/CEP-MSA Complex

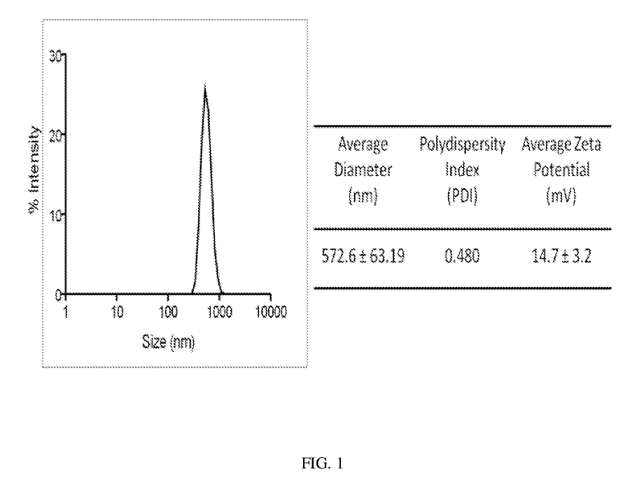

DLS studies show an average diameter of approximately 600 nm (FIG. 1). These studies show no concern of disparity in size and were tested within 24 hours at room temperature as well as after 48 hours at 4° C. A modest positively charged PDD/cargo complex is purposely tailored and optimized by calibrating the ratio of PDD to cargo. It is postulated that reducing the positive charge has two effects on optimal targeting, first shuttling the complex into APCs, and second by eliminating cell cytotoxicity of dendrimer/cargo. Similarly, an enhanced vaccine efficacy, perhaps due to overall avidity of targeting peptide for binding to MHC class II on the surface of APC, was achieved in ratios of PDD/haptenated-protein that had a reduced Zeta potential.

### Example 2—Evaluation of APC Targeting Effect of PDD/CEP-MSA and its Toxicity

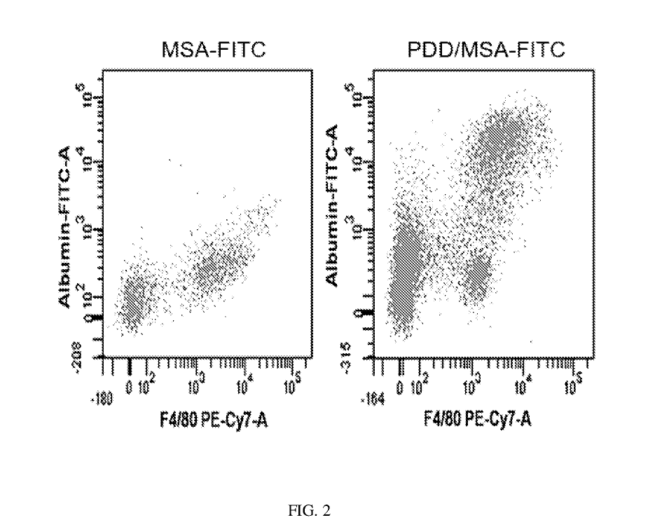

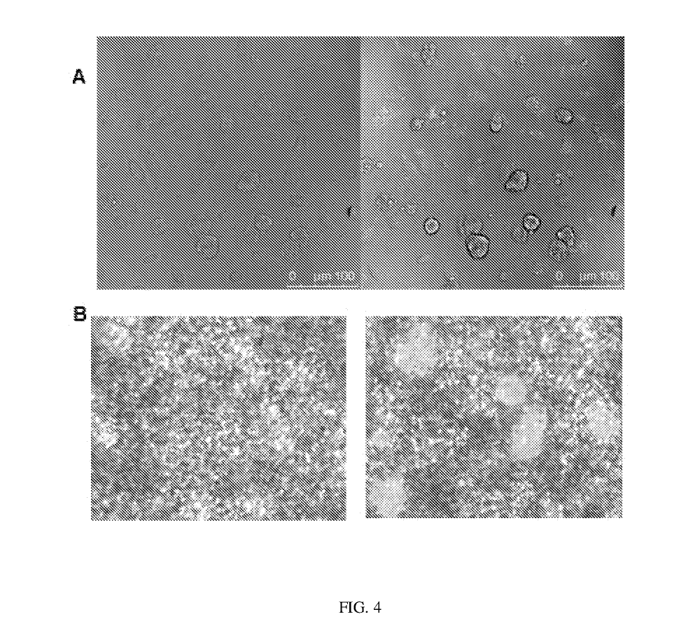

In order to determine the best ratio of PDD/Albumin-Hapten for efficient targeting of APC, intraperitoneal macrophages were collected from C578L/6 mice as described before (Daftarian et al., J Infect Dis 2013; 208(11): 1914-22). Murine macrophages were co-cultured, for 2 hours, with different weight ratios of PDD/Albumin-FITC. The ratio is a ratio of PDD to Albumin-FITC as a stand in for the ratio of PDD to albumin-hapten. For example, a ratio when albumin is used with a hapten since haptens have small MW. Cells were washed and flow cytometry analysis was performed to find optimal complex ratio results effective at targeting APCs in addition to maintaining high cell viability. The 7:1 ratio was selected for further PDD/Albumin-FITC complex formation as this ratio produced the highest cell viability as well as highest transfection efficacy (Table 2 and FIG. 2). In addition, further in vitro targeting studies using primary macrophages and fluorescent confocal microscopy imaging of labeled MSA with and without labeled PDD showed that the PDD complex was localized inside the cells (FIG. 4). This data follows previous data demonstrating the high transfection ability and buffering effect of PDD, both of which contribute to internalization of antigens allowing APCs to engulf the PDD/MSA more efficiently, a process that should lead to the presentation of haptens to T helper cells by activation effect from PADRE. For assessing in vivo targeting efficiency, mice received intraperitoneal injections of 7:1 ratio of PDD/CEP-MSA-FITC or controls. Intraperitoneal macrophages were removed. Flow cytometry analysis of these macrophages revealed that PDD targeted MSA-FITC into APCs effectively at 2 hours after injection.

TABLE 2
Table 2: Determination of optimal ratio of PDD/MSA-CEP for
targeting of APC in vitro. Intraperitoneal macrophages from C57BL/6
mice were co-cultured with various (weight) ratios of PDD/MSA-CEP-
FITC in-vitro followed by flow cytometry analysis to find optimal
complexation ratio for effective targeting of APC with
maintaining high cell viability.

| PDD:albumin- | % | % Cell |
|---|---|---|
| FITC Ratio | positive | viability |
| 0:1 | 7 (+/−2) | 92 |
| 1:1 | 18 (+/−5) | 87 |
| 1:3.5 | 22 (+/−3) | 88 |
| 1:7 | 41 (+/−7) | 87 |
| 1:14 | 44 (+/−8) | 82 |

The in vitro toxicity of PDD/cargo on human cells was studied previously and reported where it was shown that the toxicity of PDD-conjugated LAmB (Lyposmal Amphoetricin 8) on HEP G2 cells (Human liver cell) is less than the toxicity of Lamb alone. Referring to FIG. 2, an in vivo evaluation of the APC targeting effect of PDD/CEP-MSA was performed. In this evaluation, intraperitoneal macrophages were collected post IP injection of Albumin-FITC or PDD/MSA-CEP-FITC, and were analyzed by flow cytometry. Flow cytometry analysis of intraperitoneal APC showed that PDD effectively and selectively delivers MSA-CEP to APC effectively 2 hours after injection, which is compared with that of MSA-CEP-FITC alone. F4/80 is a smurine macrophage I monocyte marker.

### Example 3—PDD/CEP-MSA Induced Strong Humoral Responses

CEP-MSA was selected as a Hapten-Carrier for assessing the efficiency of PDD to induce humoral response. Mice were divided into 3 groups and each group was immunized by a different adjuvant method: PDD, Titermax and No adjuvant. The same doses were used for the initial dose, the booster at 2 weeks, and the final dose at day 25. Total Serum IgG against CEP-MSA was measured by indirect ELISA assays, total IgG induced by PDD was more than Titermax and CEP-MSA without adjuvant. This result shows that antigen delivery to APCs by the PADRE conjugated dendrimer induces a strong humoral response.

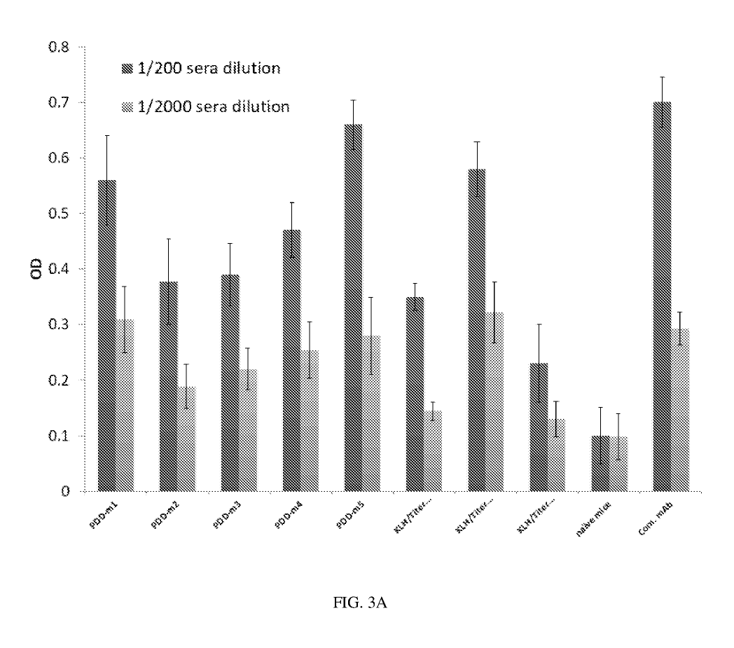

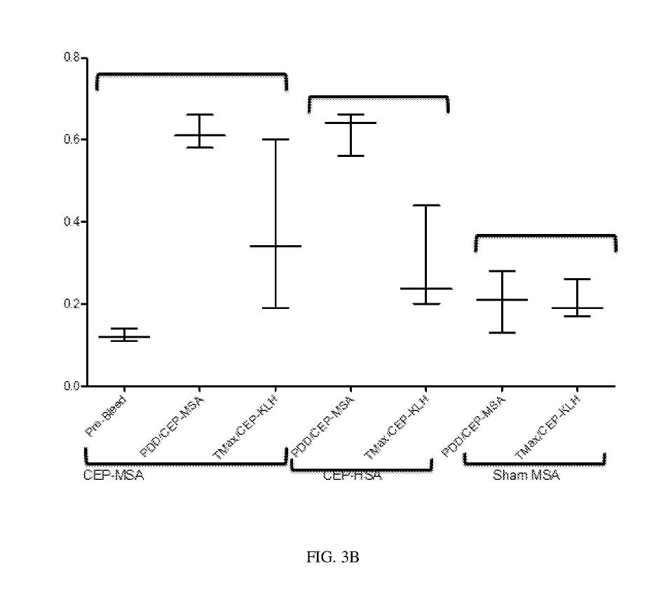

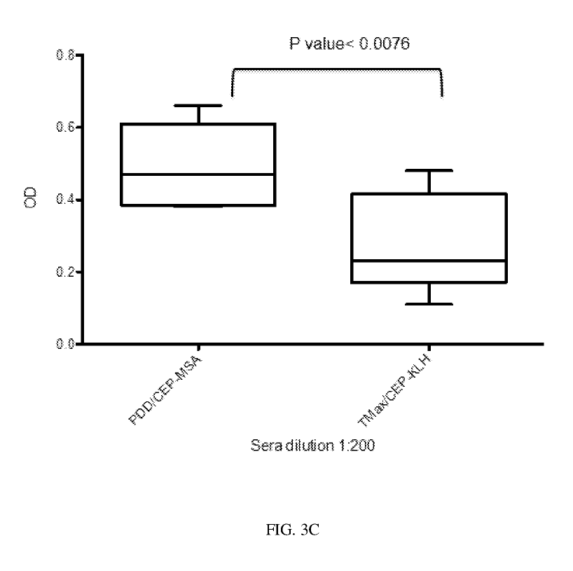

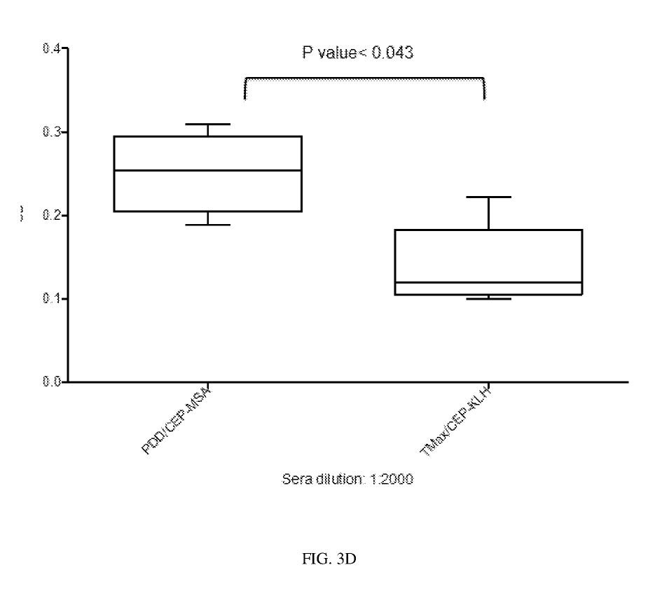

Elicitation of humoral responses against haptens is a challenging task for they are poorly immunogenic even when co-administered with adjuvants. To correct the poor immunogenicity, haptens need to be covalently coupled to a “non-self” carrier to induce immunologic responses. Unfortunately, non-self carriers such as KLH or Tetanus toxin elicit overwhelming immunologic response against their own epitope instead of haptens coupled to them. On the other hand, “self” carriers such as self-albumin are less immunogenic but usually cannot induce a strong humoral response and need adjuvants such as Alum, Titermax, or IFA, most of which have safety and regulatory issues and they may raise nonspecific cross-reacting antibodies due to their general stimulatory effect on concurrent immune reactions. Thus, there is great utility and a great need for in designing and developing a vaccine platform that can induce production of a strong and specific immunoglobulin against hapten. A platform as described herein can include G5 dendrimer-PADRE complexed to a self-protein, albumin, which is decorated with a hapten. In order to evaluate the specificity of humoral response elicited by PDD, sera of mice immunized with PDD/CEP-MSA, CEP-MSA alone and CEP-MSA with Titermax were tested in a series of ELISAs for their reactivity against CEP-MSA or MSC-SHAM (albumin processed through antigen conjugation process without adding CEP) where an anti-CEP monoclonal antibody (anti-CEP mAb) served as a positive control. The polyclonal activity of the sera of the PDD/MSA immunized mice was significantly higher than those immunized with CEP-MSA/Titermax and CEP-MSA alone. Also, in addition to a higher total amount of anti-CEP antibody, PDD had increased antibody specificity against CEP-MSA (higher OD ratio of antibody reactivity against CEP-MSA and SHAM) when compared to Titermax and CEP-MSA alone. This is probably because the stable complex formation of hapten (in CEP-MSA) to PDD results in more efficient antigen delivery to APCs as well as T helper cell activation (via PADRE) in different steps of the humoral response. Also, as shown in FIG. 3, comparable titers of anti-CEP and a similar OD ratio of antibody binding to CEP-MSA and SHAM were demonstrated in the sera of mice immunized by Titermax and CEP-MSA alone. This indicates that Titermax increases total IgG (antigen specific and cross reacting antibodies) in mice and it did specifically raise modest antibody against CEP, albeit significantly lower than PDD. Comparing the OD ratio of antibody reactions against CEP-MSA and SHAM in PDD-immunized mice and commercial monoclonal antibodies showed that immunization with PDD/CEP-MSA resulted in antisera as specific as commercial anti-CEP mAb. The OD ratio [OD CEP-MSA/OD SHAM] in PDD-induced antisera was the same as the OD ratio achieved by monoclonal antibody indicating PDD increased anti-hapten specific antibodies without increasing the antibody cross reacting with carrier (albumin).

These experiments demonstrate that PDD serves as an antigen-specific adjuvant and performs far superior to Titermax (FIG. 3) or IFA, for induction of humoral responses against haptens in haptenated protein carriers. In order to better evaluate the application of the PDD immunization as a method for PDD/CEP-monoclonal antibody KLH production and compare it to the commercial monoclonal antibody, one CEP-MSA/PDD-immunized mouse with high titers was selected and via standard methods anti-CEP mAbs were generated (Daftarian et al., Hybridoma (Larchmt) 2011; 30(5): 409-18). After 2 immunizations followed by a final intraperitoneal (i.p.) injection of (50 ug of CEP-MSA) booster, two anti-CEP clones with the highest titers were selected from CEP-MSA/PDD-immunized mice.

For a precise comparison between mAb developed by conventional immunization methods and mAb developed by PDD as described herein, an ELISA assay of mAb developed by PDD methodology described herein and commercial mAb against CEP-MSA, CPP-MSA (CPP has similar structure to CEP), SHAM and CEP-HSA (Horse Serum Albumin) was performed. The specificity of antibody recognition of hapten epitope, discrimination of carrier epitope from hapten epitope, and consistent binding of the resulting mAb to epitope, regardless of the carrier bound to the epitope, was evaluated. The result confirmed that mAb developed by the PDD method described herein was more specific than commercial mAb developed by conventional immunization methods based on the following assessed parameters. First, PDD-based mAbs show a lower background interaction to self-carrier than that of a commercial antibody made by conventional non-self carriers. A greater OD ratio of CEP-MSA to SHAM binding (OD CEP-MSA ELISA/OD SHAM ELISA) was observed by the mAbs generated by PDD methodology compared to that of commercial mAb. This revealed that mAb generated by PDD methodology can discriminate hapten epitope from carrier epitope even better than commercial mAb. Second, PDD-generated mAbs show more CEP specificity than that of a commercial antibody made by conventional non-self carriers. A greater OD ratio of CEP-MSA to CPP-MSA binding of PDD induced mAbs in comparison to similar ratios of commercial mAb demonstrated that PDD mAb can precisely recognize hapten epitope regardless of the small size of hapten and presence of the counterpart with similar structure (CPP). Third, a smaller OD ratio of CEP-MSA to CEP-HSA binding of PDD induced mAb in comparison to the same OD ratio of commercial mAb confirmed that PDD methodology produces mAb, which binds consistently, and strongly to hapten regardless of the native hapten-carrier or species. This is important for translational studies moving from preclinical vaccine studies to larger animals or to human.

### Example 4—Vaccine Platform

To perform proof of principal and to examine the potency of PDD/protein-adduct formulation as a hapten vaccine platform, mouse albumin adducts of CEP were made. The hapten used in this study, CEP, is a hapten involved in the pathogenesis of some inflammatory-mediated diseases including AMD and is a byproduct generated from the oxidation of the omega-3 fatty acid docosahexaenoic (DHA) acid in the retina. There is a pressing need for i) a reliable animal model to better develop treatments for preventing the progression of AMD, and ii) accurate lab-based correlates/surrogates of the disease progression in the laboratory. Photoreceptors in the retina contain a high level of DHA phosopholipids. In the presence of light and oxygen, DHA oxidizes, forming a reactive chemical species (HOHA) that forms a CEP on the terminal amino group of protein residues, a “CEP adduct”. Mouse serum albumin as a syngeneic protein was used since it has been shown that albumin complexes with dendrimer using its negatively charge pockets. PDD/CEP-MSA complexes were characterized and optimized for a ratio that has the highest APC targeting without cell toxicity, where the targeting studies was performed both in vitro and in vivo. The PDD/CEP-MSA was then compared with CEP-KLH formulated in Titermax for the elicitation of anti-CEP antibodies. In these studies, mice received 20 ug of CEP adducts with PDD versus 50 ug of CEP-KLH and yet the antibody responses elicited with PDD formulations were significantly superior. Next, a PDD/CEP-MSA immunized mice was selected and fusions were performed to generate anti-CEP mAbs. Three IgG1 clones were selected and were compared with an anti-CEP clone that was made by injections of IFA/CEP-KLH (commercial mAb). Two of the three PDD generated clones were more specific than the commercial mAbs.

These data suggest that PDD is a safe and adjuvanted vaccine-carrying nanoplatform to eliminate the need for the protein carriers that normally contains strong immunogenic epitopes that take over immune responses undermining the responses against the real target, the hapten. Likewise, PDD jettisons the need for Incomplete Freund's Adjuvant (IFA), which is associated with documented animal discomfort and therefore animal committee and IACUC offices strongly oppose the use of CFA. Also, these data show that PDD delivers hapten-self protein complexes in vivo and elicits humoral immune responses superior to that of the same antigen formulated in Titermax. The PDD platform targets APCs in the host resulting in a lowered antigen needed and provides universal T helper epitope for the haptens and is otherwise void of immunodominant interfering epitopes.

In summary, a CEP-MSA/PDD vaccine platform was created that can be used to elicit highly specific anti-CEP antibody responses. This platform negates the use of non-self immunodominant immunogenic carriers, which elicit overwhelmingly immune responses resulting in strong immune responses, e.g. generating many antibodies, against the carrier and not the hapten. Furthermore, such potent hapten-irrelevant carriers are known to potentially suppress anti-hapten immune responses. Since the PDD platform has APC targeting ability, it generates higher value hapten-specific antibodies with higher specificity and lowers the dose and the frequency of immunizations.

### OTHER EMBODIMENTS

Any improvement may be made in part or all of the compositions, conjugates, vaccines, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. For example, although the experiments described herein involve CEP as the hapten, the compositions, conjugates, vaccines, kits, and methods described herein can be used to generate a strong humoral response against any hapten or other poorly immunogenic antigen of interest. Similarly, although the experiments described herein involved PDD, in addition to PADRE, any suitable T helper peptide can be used. Any statement herein as to the nature or benefits of the invention or of preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A conjugate comprising at least one charged dendrimer having conjugated thereto: a) at least one T helper peptide that specifically binds to a professional antigen presenting cell (APC), b) at least one hapten or antigen of low immunogenicity, and c) at least one syngeneic peptide or protein.

2. The conjugate of claim 1, wherein the subject is a mammal.

3. The conjugate of claim 1, wherein the at least one T helper peptide is a Pan-DR epitope (PADRE).

4. The conjugate of claim 1, wherein the at least one T helper peptide comprises the amino acid sequence of any of SEQ ID NOs: 1-33 or a derivative thereof.

5. The conjugate of claim 1, wherein the at least one charged dendrimer is a PAMAM dendrimer.

6. The conjugate of claim 2, wherein the syngeneic peptide or protein is serum albumin.

7. A method of producing antibodies against a hapten or antigen of low immunogenicity in a subject comprising the steps of:
a) immunizing the subject with the conjugate of claim 1 resulting in antibodies specific for the at least one hapten or antigen of low immunogenicity; and
b) isolating the antibodies.

8. The method of claim 7, wherein the antibodies are polyclonal antibodies.

9. A method of producing monoclonal antibodies against a hapten or antigen of low immunogenicity in a subject comprising immunizing the subject with the conjugate of claim 1 resulting in reactive B cells for making monoclonal antibodies via fusions and generation of hybridomas, via phage display technology, or via any manipulation of B cell nucleic acids.

10. A method of increasing immunogenicity of a hapten or antigen of low immunogenicity in a subject comprising conjugating the hapten or antigen of low immunogenicity to a charged dendrimer having conjugated thereto: a) at least one T helper peptide that specifically binds to a professional APC, and b) at least one syngeneic peptide or protein.

11. A vaccine for eliciting a humoral response against a hapten or antigen of low immunogenicity in a subject comprising the conjugate of claim 1 and a pharmaceutically acceptable carrier.

12. A kit for generating antibodies against a hapten or antigen of low immunogenicity, the kit comprising:
a) a plurality of conjugates, each conjugate comprising at least one charged dendrimer having conjugated thereto: a) at least one T helper peptide that specifically binds to a professional APC, b) at least one hapten or antigen of low immunogenicity, and c) at least one syngeneic peptide or protein;
b) instructions for use; and
c) packaging.
